(19) United States
(12) Patent Application Publication
(10) Pub. No.: US 2018/0232490 A1
(43) Pub. Date: Aug. 16, 2018

(54) ACCESSING DATA FROM MULTIPLE DIFFERENT SOURCES

(71) Applicant: Microsoft Technology Licensing, LLC, Redmond, WA (US)

(72) Inventors: Rouella J. Mendonca, Redmond, WA (US); Victor Vuong, Kirkland, WA (US); Abhijeet Thacker, Bothell, WA (US)

(21) Appl. No.: 15/605,675

(22) Filed: May 25, 2017

Related U.S. Application Data
(60) Provisional application No. 62/459,885, filed on Feb. 16, 2017.

(51) Int. Cl. G06F 19/00; A61B 5/021; A61B 5/11

(52) U.S. Cl. CPC G06F 19/322; A61B 5/021; G06F 19/328; G06F 19/325; G06F 19/3481; A61B 5/1118

(57) ABSTRACT

In example embodiments, a machine receives a medical record of a patient. The machine receives, from devices associated with the patient, activity data and physiological data related to the patient. The machine normalizes the activity data and the physiological data. The machine determines adherence to one or more tasks in a treatment plan based on the medical record, the activity data, and the physiological data by comparing the medical record, the activity data or the physiological data with one or more threshold values, the one or more threshold values residing in a medical data repository or being provided from a computing device of a health professional. The machine selects, for provision to a health professional, a portion of the medical record, a portion of the activity data, and a portion of the physiological data based on a specification provided by the health professional and permission provided by the patient.

### PRIORITY CLAIM

This application claims priority to U.S. Provisional Patent Application No. 62/459,885, filed on Feb. 16, 2017, and titled “COMPUTING DEVICES FOR MONITORING PATIENT TREATMENT PLANS,” the entire disclosure of which is incorporated herein by reference.

### RELATED APPLICATIONS

This application relates to claims priority to U.S. Patent Application No. ______ filed on , having attorney docket no. 1777.058US1 and titled “COMPUTING DEVICES FOR MONITORING PATIENT TREATMENT PLANS,” the entire disclosure of which is incorporated herein by reference. This application relates to claims priority to U.S. Patent Application No. ______, filed on ______, having attorney docket no. 1777.059US1 and titled “BROKERING DATA TO INTERESTED PARTIES,” the entire disclosure of which is incorporated herein by reference. This application relates to claims priority to U.S. Patent Application No. ______, filed on ______, having attorney docket no. 1777.061US1 and titled “ARTIFICIAL INTELLIGENCE TO EDIT HEALTH CARE PLANS,” the entire disclosure of which is incorporated herein by reference.

### BACKGROUND

A health professional may provide a treatment plan (e.g. a diet or exercise plan) for a patient. However, after the patient leaves the health professional's office, the health professional has no way to monitor the patient's compliance with the plan. The health professional and the patient may communicate with one another using computing devices, for example, via an email, instant messaging, or voice-calling program.

### BRIEF DESCRIPTION OF THE DRAWINGS

Some embodiments of the technology are illustrated, by way of example and not limitation, in the figures of the accompanying drawings.

FIG. 1 illustrates an example system in which patient treatment plans may be monitored, in accordance with some embodiments.

FIG. 2 is a data flow diagram for an example of remote monitoring, in accordance with some embodiments.

FIG. 3 is a data flow diagram for an example of brokering data to interested parties, in accordance with some embodiments.

FIG. 4 is a data flow diagram for an example of accessing data from multiple different sources, in accordance with some embodiments.

FIG. 5 is a data flow diagram for an example of using artificial intelligence to edit treatment plans, in accordance with some embodiments.

FIG. 6 is a flow chart illustrating an example method for remote monitoring, in accordance with some embodiments.

FIG. 7 is a flow chart illustrating an example method for brokering data to interested parties, in accordance with some embodiments.

FIG. 8 is a flow chart illustrating an example method for accessing data from multiple different sources, in accordance with some embodiments.

FIG. 9 is a flow chart illustrating an example method for using artificial intelligence to edit treatment plans, in accordance with some embodiments.

FIGS. 10A-10B illustrate an example user interface for identifying data types for a health professional to access, in accordance with some embodiments.

FIG. 11 is a block diagram illustrating components of a machine able to read instructions from a machine-readable medium and perform any of the methodologies discussed herein, in accordance with sonic embodiments.

### SUMMARY

The present disclosure generally relates to machines configured for monitoring patient treatment plans, including computerized variants of such special-purpose machines and improvements to such variants, and to the technologies by which such special-purpose machines become improved compared to other special-purpose machines that provide technology for monitoring patient treatment plans. In particular, the present disclosure addresses systems and methods for monitoring patient treatment plans.

According to some aspects, a machine receives, from a computing device of a health professional, a treatment plan for a patient. The machine receives, from a plurality of devices associated with the patient, activity data related to the patient. The machine determines that the activity data is related to compliance with the treatment plan. The machine provides, in response to determining that the activity data is related to compliance with the treatment plan, the activity data to the computing device of the health professional.

According to some aspects, a machine receives, from a first computing device of a first health professional, a first treatment plan for a patient. The machine receives, from a second computing device of a second health professional, a second treatment plan for the patient. The machine receives, from a plurality of devices associated with the patient, activity data related to the patient. The machine stores, in a data repository, the first treatment plan, the second treatment plan, and the activity data. The machine receives, from one device from among the plurality of devices associated with the patient, an identification of information to be provided to the first health professional and an identification of information to be provided to the second health professional. The machine provides activity data to the first computing device of the first health professional based on the activity data being related to the first treatment plan and based on the identification of information to be provided to the first health professional. The machine provides activity data to the second computing device of the second health professional based on the activity data being related to the second treatment plan and based on the identification of information to be provided to the second health professional.

According to some aspects, a machine receives a medical record of a patient. The machine receives, from a plurality of devices associated with the patient, activity data and physiological data related to the patient. The machine determines adherence to one or more tasks in a treatment plan based on the medical record, the activity data, and the physiological data. The machine selects, for provision to a health professional, a portion of the medical record, a portion of the activity data, and a portion of the physiological data based on a specification provided by the health professional and permission provided by the patient. The machine provides, to a computing device of a health professional, an indication of adherence to the one or more tasks in the treatment plan, the selected portion of the medical record, the selected portion of the activity data, and the selected portion of the physiological data.

According to some aspects, a machine receives, from a computing device of a health professional, a treatment plan for a patient, the treatment plan comprising a plurality of tasks and a goal. The machine receives, from a plurality of devices associated with the patient, activity data and physiological data related to the patient. The machine determines, based on the activity data and the physiological data, the patient's compliance with one or more tasks in the treatment plan. The machine updates the treatment plan based on the patient's compliance with the one or more tasks.

### DETAILED DESCRIPTION

Overview

The present disclosure describes, among other things, methods, systems, and computer program products that individually provide various functionality. In the following description, for purposes of explanation, numerous specific details are set forth in order to provide a thorough understanding of the various aspects of different embodiments of the present disclosure. It will be evident, however, to one skilled in the art, that the present disclosure may be practiced without all of the specific details.

As noted above, a health professional may provide a treatment plan (e.g. a diet or exercise plan) for a patient. However, after the patient leaves the health professional's office, the health professional has no way to monitor the patient's compliance with the plan, or to revise the plan based on the patient's compliance data. Computing devices for monitoring the patient's compliance with the treatment plan may be desirable. In addition, computing devices that provide artificial intelligence for suggesting changes to the treatment plan based on the patient's compliance may be desirable. As used herein, a health professional may be any person involved in the healthcare of a patient, such as a physician, a nurse, a physical therapist, a trainer, a scientist, a psychiatrist, a psychologist, a healthcare consultant and the like.

Some aspects of the subject technology involve collecting personal information associated with a user of a computing device (e.g., a patient). It should be noted that the personal information about a user may be collected after receiving affirmative consent from the users for the collection and storage of such information. Persistent reminders (e.g., email messages or information displays within an application) may be provided to the user to notify the user that his/her information is being collected and stored. The persistent reminders may be provided whenever the user accesses an application or once every threshold time period (e.g., an email message every week). For instance, an arrow symbol may be displayed to the user on his/her mobile device to notify the user that his/her global positioning system (GPS) location is being tracked. Personal information is stored in a secure manner to ensure that no unauthorized access to the information takes place. For example, medical and health related information may be stored in a Health Insurance Portability and Accountability Act (HIPAA) compliant manner in the United States, or in a manner that complies with similar laws and privacy regulations in other jurisdictions.

Some embodiments of the subject technology relate to cloud health services. According to some aspects, a server receives, from a computing device of a health professional, a treatment plan for a patient. The server receives, from a plurality of devices associated with the patient, activity data related to the patient. The devices associated with the patient may include one or more of a sensor, an activity tracker, a mobile phone, a tablet computer, a laptop computer, a desktop computer, and the like. The machine determines that the activity data is related to compliance with the treatment plan. The machine provides, in response to determining that the activity data is related to compliance with the treatment plan, the activity data to the computing device of the health professional. Some information related to the patient, such as the patient's medical record(s), treatment plan(s), and activity data may be stored in a data repository accessible via a network.

According to some aspects, a server receives, from a first computing device of a first health professional, a first treatment plan for a patient. The server receives, from a second computing device of a second health professional, a second treatment plan for the patient. The server receives, from a plurality of devices associated with the patient, activity data related to the patient. The server stores, in a data repository, the first treatment plan, the second treatment plan, and the activity data. The data repository may be a HIPAA-compliant data repository. The server receives, from one device from among the plurality of devices associated with the patient, an identification of information to be provided to the first health professional and an identification of information to be provided to the second health professional. The server provides activity data to the first computing device of the first health professional based on the activity data being related to the first treatment plan and based on the identification of information to be provided to the first health professional. The server provides activity data to the second computing device of the second health professional based on the activity data being related to the second treatment plan and based on the identification of information to be provided to the second health professional.

According to some aspects, a server receives a medical record of a patient. The server receives, from a plurality of devices associated with the patient, activity data and physiological data related to the patient. The server determines adherence to one or more tasks in a treatment plan based on the medical record, the activity data, and the physiological data. The server selects, for provision to a health professional, a portion of the medical record, a portion of the activity data, and a portion of the physiological data based on a specification provided by the health professional and permission provided by the patient. The server provides, to a computing device of a health professional, an indication of adherence to the one or more tasks in the treatment plan, the selected portion of the medical record, the selected portion of the activity data, and the selected portion of the physiological data.

According to some aspects, a server receives, from a computing device of a health professional, a treatment plan for a patient, the treatment plan includes a plurality of tasks (e.g. running on a treadmill for 30 minutes, weightlifting, and the like) and a goal (e.g. reaching a target heart rate). The server receives, from a plurality of devices associated with the patient, activity data and physiological data related to the patient. The server determines, based on the activity data and the physiological data, the patient's compliance with one or more tasks in the treatment plan. The server updates the treatment plan based on the patient's compliance with the one or more tasks.

### Example Implementations

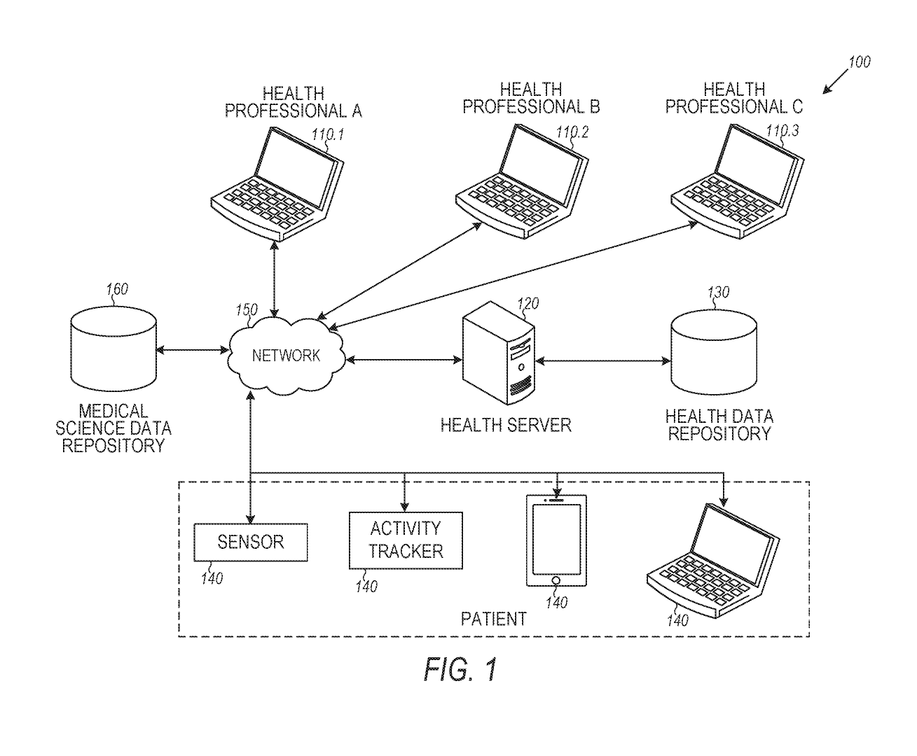

FIG. 1 illustrates an example system 100 in which patient treatment plans may be monitored, in accordance with some embodiments. As shown, the system 100 includes computing devices 110 of health professionals, a health server 120, a health data repository 130, a medical science data repository 160, and computing devices 140 of a patient. As shown, the computing devices 110 of health professionals include three computing devices 110.1-3 of three different health professionals A, B, and C, who are working with the patient. However, the subject technology may be implemented with a different number of health professional's computing devices and health professionals. As shown, the computing devices 140 of the patient include a sensor, an activity tracker, a mobile phone, and a laptop computer (which may he portable). However, the computing devices 140 of the patient may include other or different devices. The health data repository 130 stores health information about multiple patients, including the patient associated with the computing devices 140. The health data repository 130 is accessible via the health server 120, which may include software or hardware to comply with HIPAA or similar laws. The health data repository 130 may be a database or any other data storage unit.

The medical science data repository 160 stores medical information similar to information found in a physician's desk reference (or similar books). For example, the medical science data repository 160 stores information about healthy and unhealthy measurements (e.g., heart rate, blood pressure, and the like) for patients of various heights, weights, ages, genders, and medical conditions. The information in the medical science data repository 160 may be obtained from an aggregate analysis of data in the health data repository 130, while maintaining patient privacy, or from a physician's desk reference book. The medical science data repository 160 may be a database or any other data storage unit. The network 150 allows the machines 110, 120, 160, and 140 to communicate with one another. The network 150 may include one or more of the Internet, an intranet, a local area network, a wide area network, a wired network, a wireless network, a cellular network, a Wi-Fi network, a virtual private network, and the like.

According to some embodiments, a health professional, such as Health professional A, creates a treatment plan (e.g. a diet or exercise plan, which may include physiological goals) for the patient at the computing device 110.1. The health professional transmits the treatment plan, via the network 150, to the health server 120 for storage in the health data repository 130. The health professional also transmits the treatment plan to one or more of the patient computing devices 140 for monitoring and tracking the patient's compliance with the treatment plan. The patient computing devices 140 monitor the patient's compliance with the treatment plan based on manual entry by the patient or based on monitoring and tracking from devices such as the sensor and the activity tracker. As used herein, compliance relates to the plan. For example, if a task includes jogging for 30 minutes, at least 30 minutes of jogging (e.g., not 20 minutes) would lead to compliance. However, each task may be configured independently according to the health professional's preference. Each activity may be configured independently, and the health professional may be able to see with which tasks the patient does and does not comply. The patient computing devices 140 provide data related to the patient's compliance, via the network 150, to the health server 120 for storage in the health data repository 130. The health professional can access, from the health professional's computing device 110.1 and via the network 150 and the health server 120, data related to the patient that is stored in the health data repository 130 (including data related to compliance with the treatment plan) based on permissions provided to the health professional by the patient.

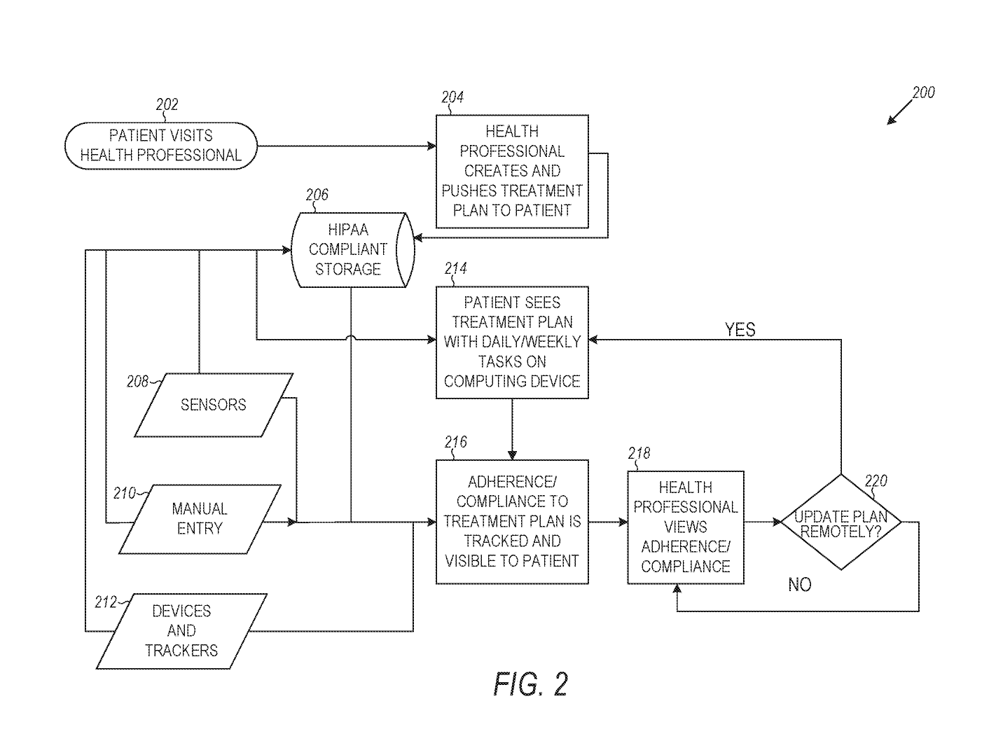

FIGS. 2-5 are data flow diagram of various processes that may be implemented in the system 100 or in other computer systems. While the processes are described as being implemented within the system 100, the processes may also be implemented in different systems, which may include different machines.

FIG. 2 is a data flow diagram for an example of a process 200 for remote monitoring, in accordance with some embodiments. As shown, at block 202, a patient visits a health professional. At block 204, the health professional creates and pushes a treatment plan to the patient. For example, the treatment plan may be pushed from the health professional's computing device 110 to the patient's computing devices 140. The treatment plan may be pushed at any time. For example, the health professional may ask the patient to open an application on the patient's computing device 140, and the application may download the treatment plan. This may occur in the health professional's office or remotely from the health professional (e.g., in the patient's home or office). The health professional could use a health professional portal (e.g., at the health professional's computing device 110) to create, update and delete plans and tasks for the patient. This plan is then pushed to the application at the patient's computing device 140, where the patient tracks compliance. The heath treatment plan is also provided to the HIPAA compliant storage 206, which may correspond to the health data repository 130. Compliance with the plan is monitored by the patient's computing devices 140, which correspond to the sensors 208, the manual entry devices 210, and the devices and trackers 212. The sensors 208, the manual entry devices 210, and the devices and trackers 212 provide information regarding compliance with the treatment plan to the HIPAA compliant storage 206. At block 214, the patient sees the treatment plan with daily/weekly tasks on the patient's computing device 140. At block 216, adherence or compliance to the treatment plan is tracked and visible to the patient via the patient's computing device 140. At block 218, the health professional, using the health professional's computing device 110, views the adherence and compliance. At block 220, the health professional may update the plan from the health professional's computing device 110. If the health professional updates the plan, the process 200 returns to block 214. If the health professional does not update the plan, the process 200 returns to block 218.

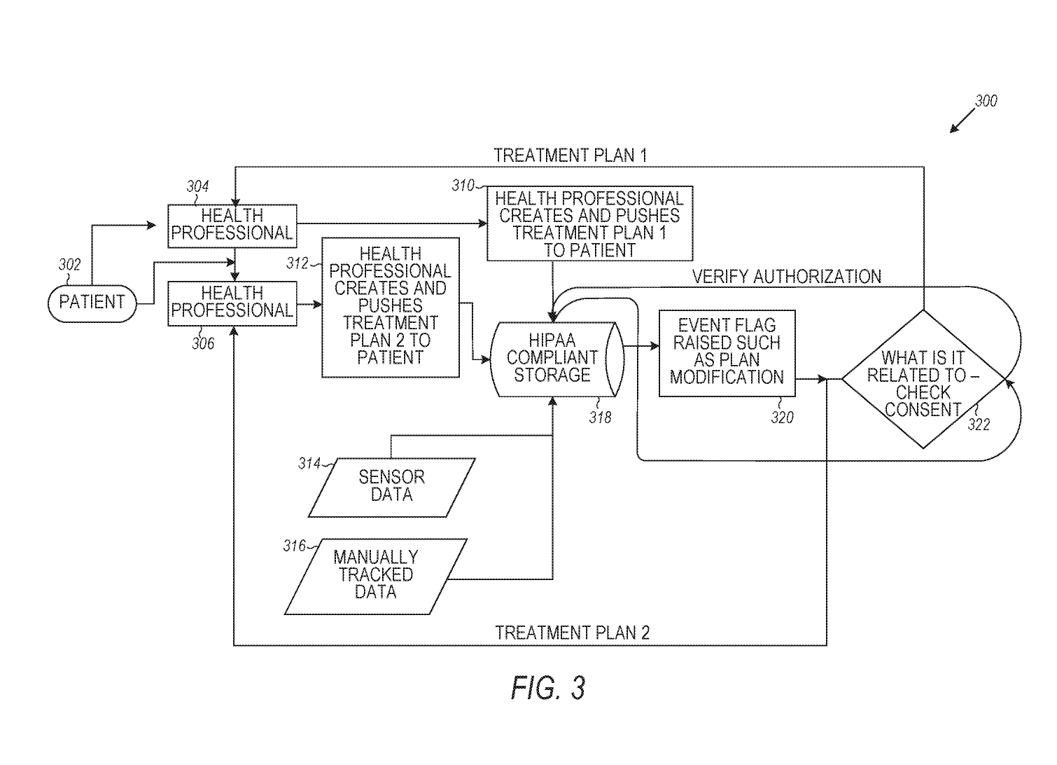

FIG. 3 is a data flow diagram for an example of a process 300 for brokering data to interested parties, in accordance with some embodiments. As shown, a patient 302 communicates with two health professionals 304 and 306. Health professional 304 creates treatment plan 1 for the patient. Health professional 306 creates treatment plan 2 for the patient. At block 310, health professional 304 pushes treatment plan 1 to the patient (e.g. to the computing devices 140 of the patient). At block 312, health professional 306 pushes treatment plan 2 to the patient (e.g. to the computing devices 140 of the patient). The treatment plans are stored in HIPAA compliant storage 318 (e.g. the health data repository 130). The HIPAA compliant storage 318 receives, from the patient's computing devices 140) sensor data 314 and manually tracked data 316 related to compliance with the treatment plans from the health professionals 304 and 306. At block 320, the HIPAA compliant storage raises an event flag when there is an event such as a plan modification. The event flag may be raised under multiple circumstances. For example, a health professional creating the plan may get an event if another health professional is given access to the plan or makes changes to a task in the plan. The event flag may be raised if a reading (e.g., blood pressure) is out of range and triggers an event. In this case, all health professionals who are interested in the reading (e.g., the patient's blood pressure) would be alerted. At block 322, the HIPAA compliant storage 318 (or an associated server, such as the health server 120) accesses the event flag and verifies authorization (e.g. provided by the patient 302) of the health professionals 304 and 306 to access the event flag. The HIPAA compliant storage 318 (or the associated server) also verifies whether the event flag is related to the treatment plan from the health professional 304 or the treatment plan from the health professional 306 based on a set of rules that are set by the health professional 304 or the health professional 306. In some examples, an event flag may be raised only if an event happens a certain number of times within a time period, for instance, if the patient's blood pressure exceeds 145 mm Hg at least three times in a 24 hour period. If the health professional 304 is authorized to access the data and the data is related to the treatment plan from the health professional 304, the HIPAA compliant storage or the associated server provides the event flag to the health professional 304. If the health professional 306 is authorized to access the data and the data is related to the treatment plan from the health professional 306, the HIPAA compliant storage or the associated server provides the event flag to the health professional 306.

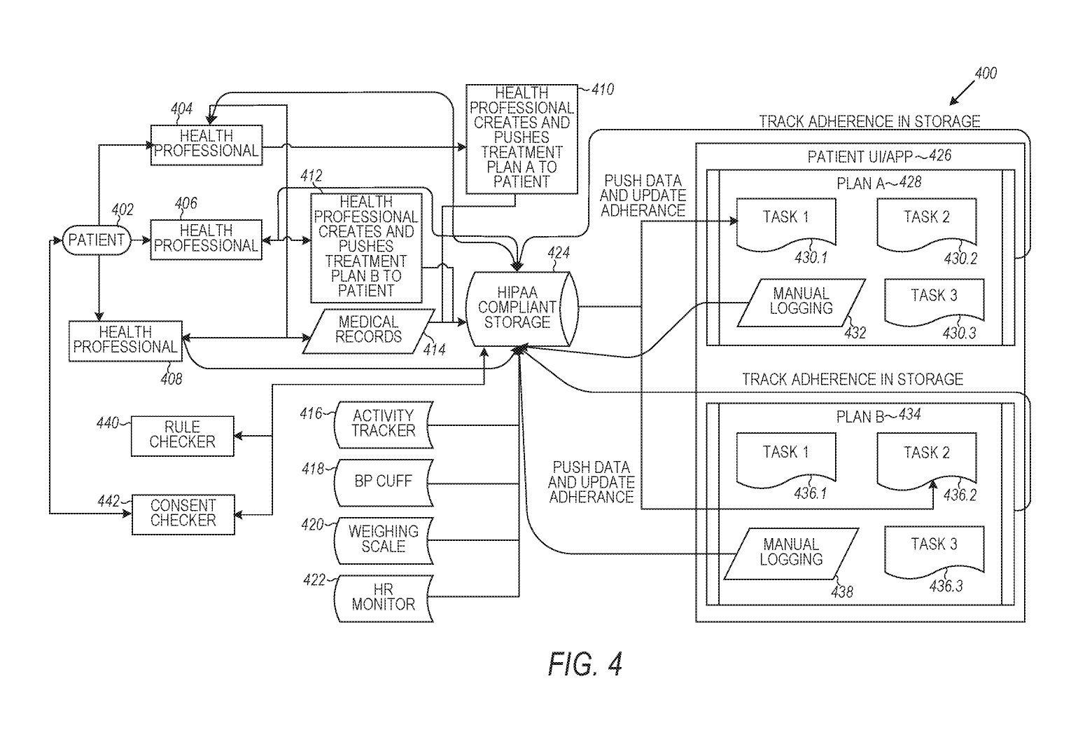

FIG. 4 is a data flow diagram for an example of a process 400 for accessing data from multiple different sources, in accordance with some embodiments. As shown, a patient 402 communicates with health professionals 404, 406, and 408. At block 410, health professional 404 creates and pushes treatment plan A 428 to the patient's computing devices 140 and the HIPAA compliant storage 424. At block 412, health professional 406 creates and pushes treatment plan B 434 to the patient's computing devices 140 and the HIPAA compliant storage 424. The patient's computing devices 140, which include the illustrated activity tracker 416, the blood pressure (BP) cuff 418, the weighing scale 420, and the heartrate (BR) monitor 422, communicate with the HIPAA compliant storage 424 regarding the patient's compliance with treatment plan A 428 and treatment plan B 434. In some cases, the patient's computing devices 140 may also include blood pressure monitors, fitness trackers, device(s) running fitness application(s), weighing scales, implantable medical devices, glucose monitoring devices, thermometers, blood oxygen sensors, and the like. The HIPAA compliant storage 424 also accesses medical records 414 related to the patient from the health professionals 404, 406, and 408. One or more of the patient's computing devices 140 presents a patient user interface (UI) application (app) 426. The patient UI app includes an interface presenting treatment plan A 428 and treatment plan B 434. As shown, treatment plan A 428 includes three tasks 430.1-3 and manual logging 432. Treatment plan B 434 includes three tasks 436.1-3 and manual logging 438. When new medical records 414 related to the tasks 430 or 436 are received at the HIPAA compliant storage 424, related tasks (e.g. tasks 430.1 and 434.2) may be updated in the treatment plans 428 and 434. For example, if the patient 402 breaks his/her leg, tasks related to walking or running (and related therapy exercises and care recommendations) may be updated. Information from the manual logging 432 and 438 is provided to the HIPAA compliant storage 424.

As shown, the HIPAA compliant storage 424 is coupled with a rule checker 440 and a consent checker 442. The rule checker 440 applies rules provided by health professionals 404, 406, and 408 before providing data from the HIPAA compliant storage 424 to the health professionals 404, 406, and 408. As a result, the health professionals 404, 406, and 408 do not receive data in which they are not interested. The consent checker 442 receives permissions from the patient 402 to provide certain data from the HIPAA complaint storage 424 to one or more of the health professionals 404, 406, and 408. The consent checker 442 determines whether data requested by one of the health professionals 404, 406 or 408 is allowed to be provided to the health professional 404, 406 or 408 based on the permissions and ensures that the health professional 404, 406 or 408 only accesses data that he/she is permitted to access by the patient 402.

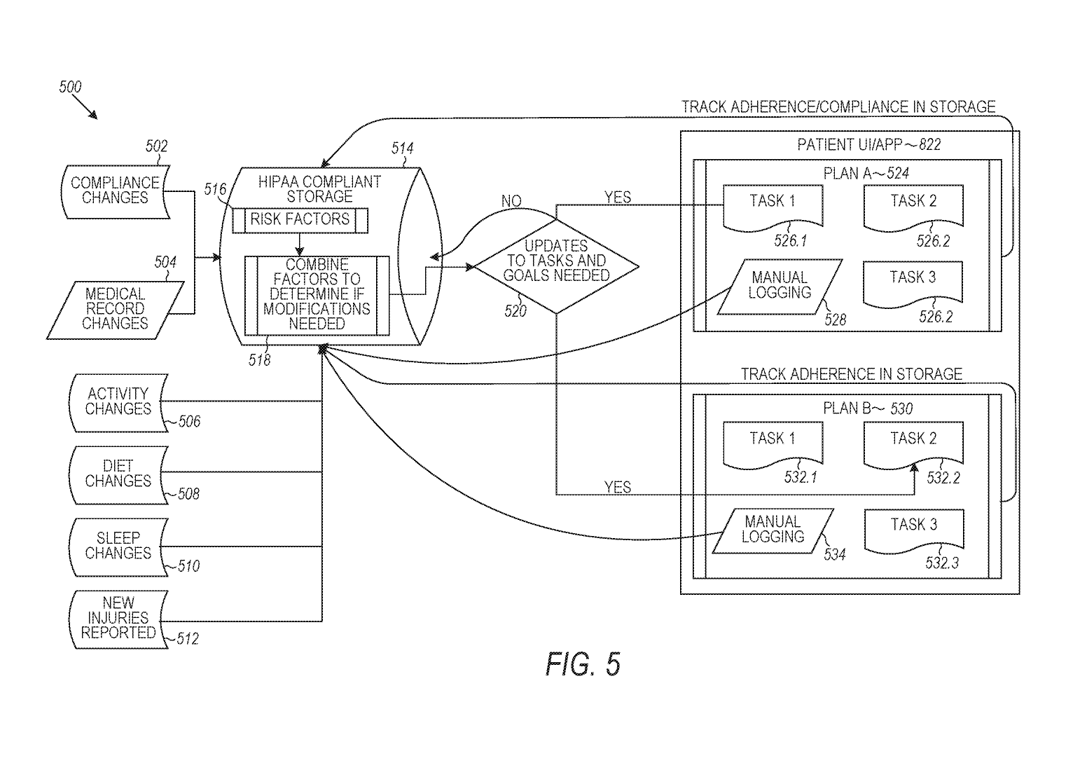

FIG. 5 is a data flow diagram for an example of a process 500 using artificial intelligence to edit treatment plans, in accordance with some embodiments. As shown in FIG. 5, compliance changes 502, medical record changes 504, activity changes 506, diet changes 508, sleep changes 510, and new injuries reported 512 are provided to HIPAA compliant storage 514 (e.g. health data repository 130). At the HIPAA compliant storage 514, risk factors 516 are combined 518 to determine if modifications to a treatment plan (e.g. treatment plan A 524 or treatment plan B 530) are needed. At block 520, a server (e.g. health server 120) determines whether updates to tasks and goals of treatment plans are needed. If so, the updates are pushed to the patient UI app 522. If not, the HIPAA compliant storage 514 is notified that no changes were made.

As shown, the patient UI app 522 includes two treatment plans for the patient-treatment plan A 524 and treatment plan B 530. Treatment plan A 524 includes three tasks 526.1-3 and manual logging 528 (e.g., of information related to compliance with the tasks 526.1-3). Treatment plan B 530 includes three tasks 532,1-3 and manual logging 534 (e.g. of information related to compliance with the tasks 532.1-3). Some of the tasks (e.g. tasks 526.1 and 532.2) may be updated via block 520 if a server (e.g. health server 120) accessing the HIPAA compliant storage 514 determines that risk factors 516 indicate that modifications to the tasks are needed.

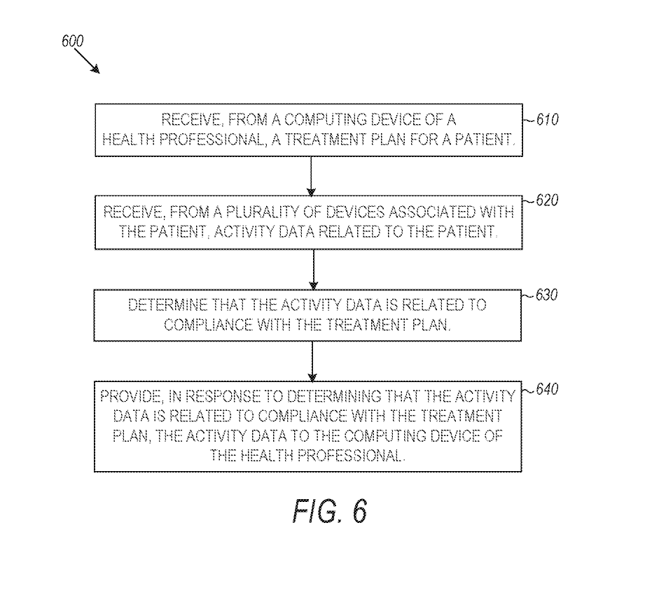

FIGS. 6-9 illustrate various methods that can be implemented in conjunction with subject technology. While the methods are discussed as being implemented using the machines of the system 100 of FIG. 1, the methods may also be implemented in other systems, which include other machines.

FIG. 6 is a flow chart illustrating an example method 600 for remote monitoring, in accordance with some embodiments. The method 600 may be implemented at the health server 120.

At operation 610, the health server 120 receives, from a computing device 110 of a health professional, a treatment plan for a patient. The treatment plan may include one or more of an exercise plan (e.g., strength training three times per week and jogging twice per week), a diet plan (e.g., eat fewer than 2000 calories per day), and a physiological goal (e.g., reach a heart rate of 170).

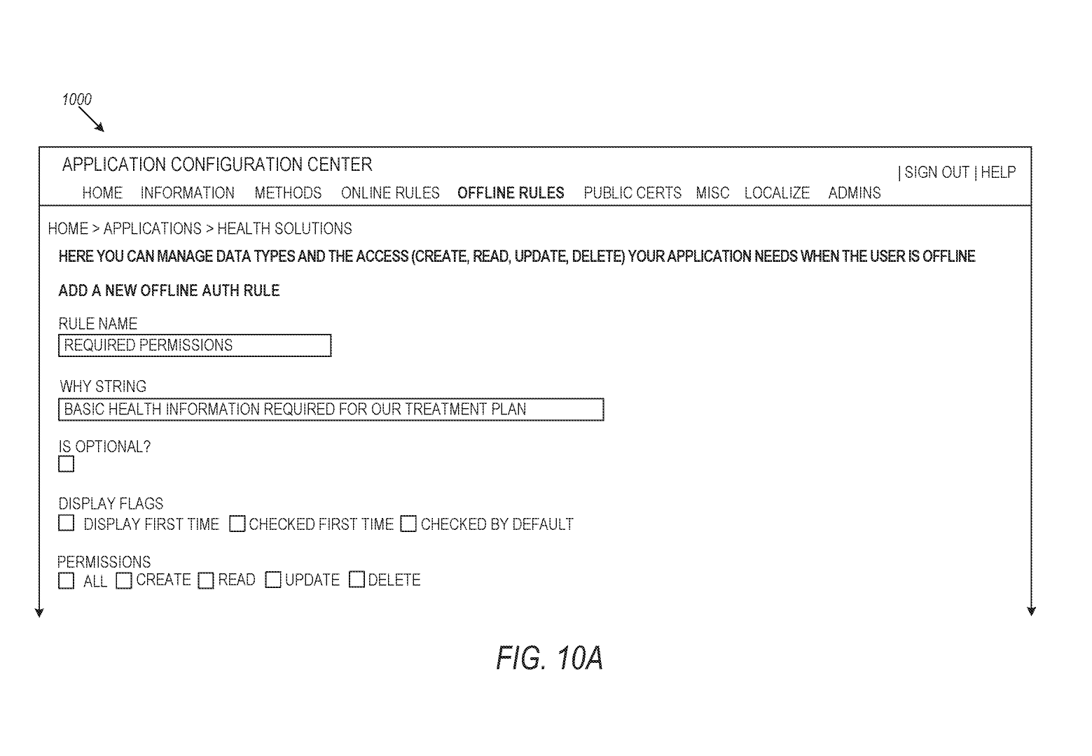

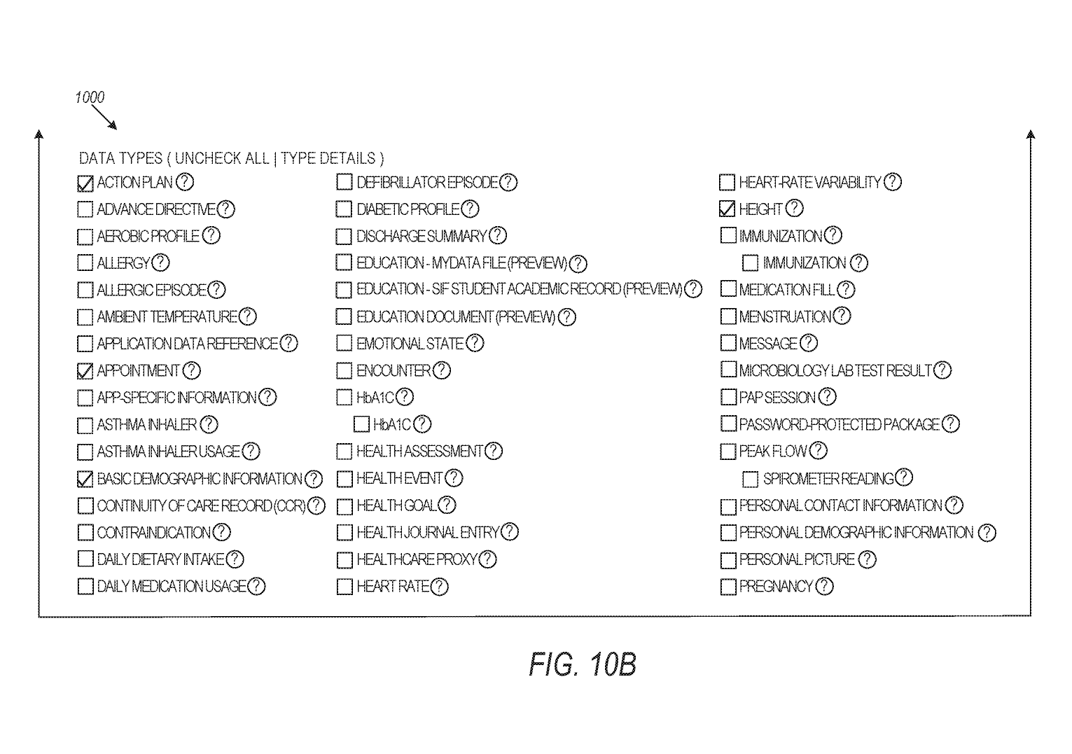

At operation 620, the health server 120 receives, from a plurality of devices 140 associated with the patient, activity data related to the patient. The activity data may be entered manually or tracked via a sensor or exercise tracker. The plurality of devices 140 may include one or more of a fitness tracker, a sensor or a computing device configured for manual entry of activity data. The computing device configured for manual entry of activity data may be a laptop computer, a desktop computer, a mobile phone or a tablet computer accessing an application or webpage through which the activity data may be manually entered. An example of such an application or webpage is described herein in conjunction with FIGS. 10A-10B. The health server 120 may store the treatment plan and the activity data related to the patient in a data repository, such as the health data repository 130.

At operation 630, the health server 120 determines that the activity is related to compliance with the treatment plan. According to sonic examples, the treatment plan is associated with one or more measureable numerical data points (e.g. a heart rate value), and the activity data corresponds to the one or more measurable numerical data points (e.g. a measurement of the heart rate). According to some examples, determining that the activity data is related to compliance with the treatment plan includes determining that the activity data corresponds to data specified, within the treatment plan, to be provided to the health professional. For example, the treatment plan may request that heart rate data be provided to the health professional. According to some examples, the health server 120 compares the activity data with threshold values for that activity data from the medical science data repository 160. For example, if the activity data is a blood pressure measurement, the blood pressure measurement may be compared with data in the medical science data repository 160 to determine if the blood pressure is normal, too high, or too low for persons of the patient's height, weight, age, gender, and medical conditions. Alternatively, the threshold values may be provided to the health server 120 from the computing device 110 of the health professional, where the health professional may tailor the measurements of the activity data about which he/she is to be notified. In some cases, the health profession may create, via the health professional's computing device 110, a rule-based system for notifications. For example, the health professional may request to receive a notification if the patient's systolic blood pressure exceeds 140 for three days in a row and exceeds 150 mmHg on at least one of the three days. In summary, the threshold values may be default values, obtained from the medical science data repository 160 based on information about the patient. Alternatively, the threshold values may be tailored to the patient by the health professional, and may be different from the values obtained from the medical science data repository 160.

At operation 640, the health server 120 provides, in response to determining that the activity data is related to compliance with the treatment plan, the activity data to the computing device 110 of the health professional. In some cases, the health server 120 receives, from one of the plurality of devices 140 associated with the patient, an indicated type of data to provide to the health professional. The health server 120 provides the activity data to the computing device 110 in response to determining that the activity data is from among the indicated type of data.

According to some implementations, the health server 120 receives, from the computing device 110 of the health professional, an update to the treatment plan generating an updated treatment plan. The health server 120 determines that the received activity data is related to compliance with the updated treatment plan. The health server 120 provides, in response to determining that the activity data is related to compliance with the updated treatment plan, the activity data to the computing device 110 of the health professional. In some cases, the treatment plan includes tasks which have schedules and reminders associated with them. Each task is stored as an independent entity in the health data repository 130, and updates to tasks and treatment plans are identified based on a unique identifier.

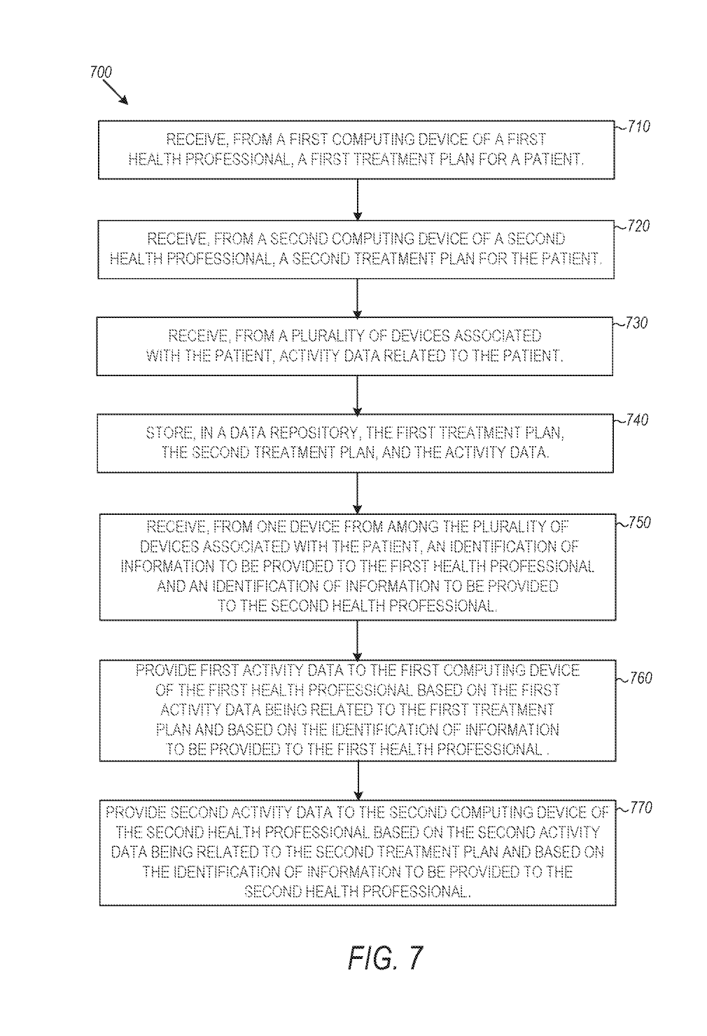

FIG. 7 is a flow chart illustrating an example method 700 for brokering data to interested parties, in accordance with some embodiments. The method 700 may be implemented at the health server 120.

At operation 710, the health server 120 receives, from a first computing device 110.1 of a first health professional (e.g. health professional A), a first treatment plan for a patient.

At operation 720, the health server 120 receives, from a second computing device 110.2 of a second health professional (e.g. health professional B), a second treatment plan for the patient.

At operation 730, the health server 120 receives, from a plurality of devices 140 associated with the patient, activity data related to the patient.

At operation 740, the health server 120 stores, in the health data repository 130, the first treatment plan, the second treatment plan, and the activity data.

At operation 750, the health server 120 receives, from one device (e.g., a mobile phone, a tablet computer, a laptop computer, or a desktop computer) from among the plurality of devices 140 associated with the patient, an identification of information to be provided to the first health professional and an identification of information to be provided to the second health professional. According to some aspects, the health server 120 provides for presentation, at the one device, of an interface for identifying the first health professional, the information to be provided to the first health professional, the second health professional, and the information to be provided to the second health professional. The health server 120 receives, via the interface, the identification of information to be provided to the first health professional and the identification of information to be provided to the second health professional.

At operation 760, the health server 120 provides first activity data to the first computing device 110.1 of the first health professional based on the first activity data being related to the first treatment plan and based on the identification of information to be provided to the first health professional. According to some implementations, identifying that the first activity data is related to the first treatment plan includes comparing, at the health server 120, the first activity data with one or more first threshold values for the first activity data. The one or more first threshold values are obtained from the first computing device 110.1 of the first health professional and are selected by the first health professional for the patient. Alternatively, the one or more first threshold values are obtained from the medical science data repository 160 based on an information tuple about the patient. The information tuple may include the patient's height, weight, age, gender and medical condition(s). In some cases, identifying that the first activity data is related to the first treatment plan includes applying, to the first activity data, a rule-based analysis provided from the medical data repository 160 or the first computing device 110.1 of the first health professional. The health profession creates, via the health professional's computing device 110.1, a rule-based system for notifications. For example, the health professional may request to receive a notification if the patient's systolic blood pressure exceeds 140 for three days in a row and exceeds 150 mmHg on at least one of the three days.

At operation 770, the health server 120 provides second activity data to the second computing device 110.2 of the second health professional based on the second activity data being related to the second treatment plan and based on the identification of information to be provided to the second health professional. According to some implementations, identifying that the second activity data is related to the second treatment plan includes comparing, at the health server 120, the second activity data with one or more second threshold values for the second activity data. The one or more second threshold values are obtained from the second computing device 110.2 of the second health professional and are selected by the second health professional for the patient.

Alternatively, the one or more second threshold values are obtained from the medical science data repository 160 based on an information tuple about the patient. The information tuple may include the patient's height, weight, age, gender and medical condition(s). In some cases, identifying that the second activity data is related to the second treatment plan includes applying, to the second activity data, a rule-based analysis provided from the medical data repository 160 or the second computing device 110.2 of the second health professional. The health profession creates, via the health professional's computing device 110.2, a rule-based system for notifications. For example, the health professional may request to receive a notification if the patient's systolic blood pressure exceeds 140 for three days in a row and exceeds 150 mmHg on at least one of the three days.

In some cases, the health server 120 provides, to the second computing device 110.2 of the second health professional, the first treatment plan for the patient based on the identification of information to be provided to the second health professional. The health server 120 receives, from the second computing device 110.2 of the second health professional, a modification to the first treatment plan. For example, if the first treatment plan includes running and the second health professional determined that the patient has a knee injury, the second health professional may modify the first treatment plan to replace running with another exercise that does not involve the knees. The health server 120 stores, in the health data repository 130, the modification to the first treatment plan. In some cases, the health server provides, to the first computing device 110.1 of the first health professional, the modification to the first treatment plan, so that the first health professional is aware of the changes. Permissions for a health professional to access health data of a patient are requested by the health professional and approved by the patient. Permissions may be granted per application, per health professional, or per organization (e.g., employer, hospital, etc.) associated with a health professional. The permissions may be stored at the health server 120 or at the health data repository 130.

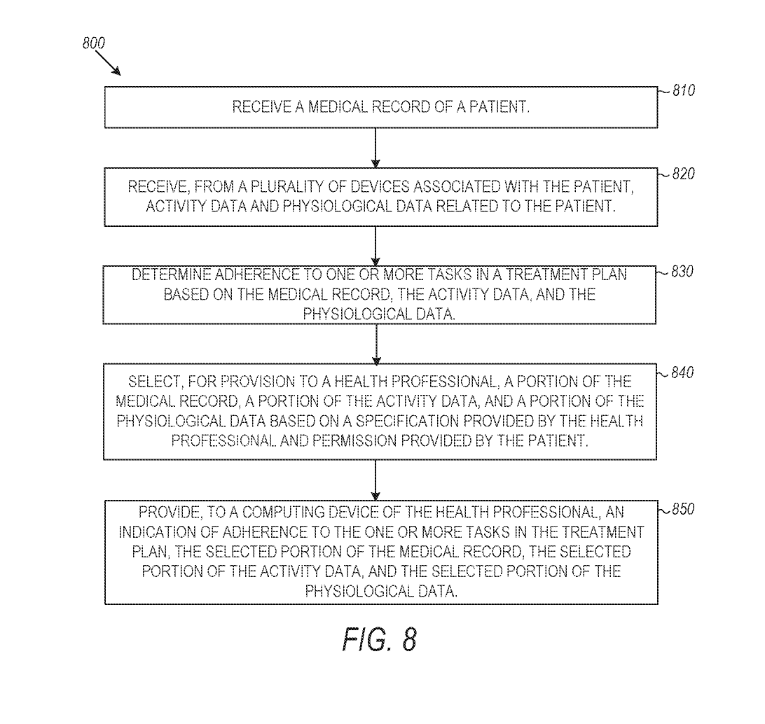

FIG. 8 is a flow chart illustrating an example method 800 for accessing data from multiple different sources, in accordance with some embodiments. The method 800 may be implemented at the health server 120.

At operation 810, the health server 120 receives, from the health data repository 130, an electronic medical record of a patient. As used herein, an “electronic medical record” may include any electronically-stored health information. For example, an electronic medical record may be a CCR (continuity of care record), a CCD (continuity of care document), or any updates to information associated with the patient and stored in the health data repository 130 (or other health data storage unit).

At operation 820, the health server 120 receives, from a plurality of devices 140 associated with the patient, activity data and physiological data related to the patient. The physiological data includes one or more of: a weight measurement, a heart rate measurement, a blood pressure measurement, a sleep measurement, an activity measurement, a cholesterol measurement, a body fat measurement, and the like. After receiving the activity data and the physiological data, the health server 120 normalizes the activity data and the physiological data. For example, a heart rate may be taken by a fitness tracker at 60 second intervals, and by a nurse at a hospital at 5 second intervals. The two heart rate measurements may be normalized (e.g., both expressed in beats per minute) so that they may be easily compared with one another and analyzed.

At operation 830, the health server 120 determines adherence to one or more tasks in a treatment plan based on the medical record, the activity data, and the physiological data. According to some examples, the treatment plan includes one or more of: a therapy plan, a disease treatment plan, an exercise plan, a diet plan, and a physiological goal. According to some examples, the treatment plan is associated with one or more measurable numerical data points (e.g. a target blood pressure measurement). The activity data corresponds to the one or more measurable numerical data points (e.g. a blood pressure measurement of the patient). In some examples, adherence is determined by comparing the numerical values associated with the one or more tasks with numerical values in the medical record, the activity data, and the physiological data. According to some examples, the health server 120 compares the medical record, the activity data, and the physiological data with threshold values from the medical science data repository 160. For example, if the physiological data is a blood pressure measurement, the blood pressure measurement may be compared with data in the medical science data repository 160 to determine if the blood pressure is normal, too high, or too low for persons of the patient's height, weight, age, gender, and medical conditions. Alternatively, the threshold values may be provided to the health server 120 from the computing device 110 of the health professional, where the health professional may tailor the measurements of the activity data about which he/she is to be notified. In some cases, the health profession may create, via the health professional's computing device 110, a rule-based system for notifications. For example, the health professional may request to receive a notification if the patient's systolic blood pressure exceeds 140 for three days in a row and exceeds 150 mmHg on at least one of the three days. In summary, the threshold values may be default values, obtained from the medical science data repository 160 based on information about the patient. Alternatively, the threshold values may be tailored to the patient by the health professional, and may be different from the values obtained from the medical science data repository 160.

At operation 840, the health server 120 selects, for provision to the health professional, a portion of the medical record, a portion of the activity data, and a portion of the physiological data based on a specification provided by the health professional and permission provided by the patient. The patient may provide permission from one of the patient's devices 140 using an interface as described, for example, in conjunction with FIGS. 10A-10B.

At operation 850, the health server 120 provides, to the computing device 110 of the health professional, an indication of adherence to the one or more tasks in the treatment plan, the selected portion of the medical record, the selected portion of the activity data, and the selected portion of the physiological data. In some cases, the health server stores the medical record, the activity data, and the physiological data in the health data repository 130.

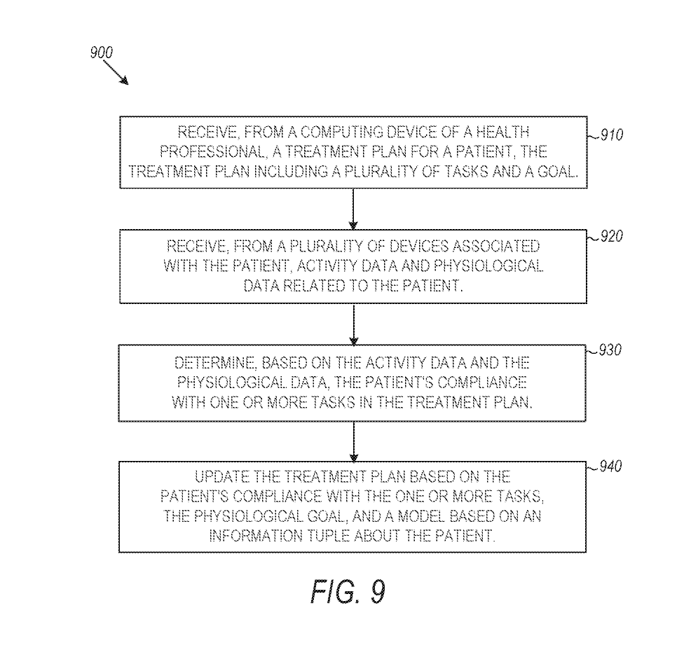

FIG. 9 is a flow chart illustrating an example method 900 for using artificial intelligence to edit treatment plans, in accordance with some embodiments. The method 900 may be implemented at the health server 120.

At operation 910, the health server 120 receives, from a computing device 110 of a health professional, a treatment plan for a patient. The treatment plan includes a plurality of tasks and a goal. The treatment plan includes one or more of: an exercise plan, a diet plan, or a physiological plan.

At operation 920, the health server 120 receives, from a plurality of devices 140 associated with the patient, activity data and physiological data related to the patient. The physiological data may include one or more of: a weight measurement, a heart rate measurement, or a blood pressure measurement. The plurality of devices 140 may include one or more of: a fitness tracker, a physiological sensor or a computing device configured for manual entry of activity data. The computing device configured for manual entry of activity data may be any device that can be coupled with a visual display unit (e.g. a screen or monitor) and a text input unit (e.g. a touchscreen or a keyboard).

At operation 930, the health server 120 determines, based on the activity data and the physiological data, the patient's compliance with one or more tasks in the treatment plan.

At operation 940, the health server 120 updates the treatment plan based on the patient's compliance with the one or more tasks, the physiological goal, and a model based on an information tuple about the patient. The information tuple may include a height (e.g., 175 cm), a weight (e.g., 75 kg), an age (e.g., 30 years old), a gender (e.g., female), and medical condition(s) (e.g., hypertension) of the patient. The model may include consulting the medical science data repository 160 the most effective treatment technique for the condition for patients having the height, weight, age, and gender of the subject patient. For example, the treatment plan may include doing 20 sit-ups and 20 push-ups every day to treat hypertension. The medical science data repository 160 may indicate that, for female patients ages 30-39 with heights of 160-180 cm and weights of 70-80 kg, sit-ups are more effective than push-ups in treating hypertension. If the patient is complying with the treatment plan, the number of sit-ups (rather than the number of push-ups) may be increased. However, if the patient is not complying with the treatment plan, the number of push-ups (rather than the number of sit-ups) may be reduced.

In some examples, the health server 120 determines that the patient is complying with the one or more task and increases an intensity of the one or more tasks. The task(s) for increasing the intensity are identified based on the goal of the treatment plan and the model based on the information tuple. For example, a treatment plan task has a blood pressure goal to be between 100 and 120 for systolic and 70 and 90 for diastolic. As part of the treatment plan, the patient is asked to measure his/her blood pressure every week. A measurement indicates that the blood pressure is above the goal range. As such the task is now updated to require a measurement every day. When the reading returns to normal, the schedule is again automatically updated to be every week. In some examples, the health server 120 determines that the patient is not complying with the one or more task and decreases an intensity of the one or more tasks or removes the one or more tasks from the treatment plan. The task(s) for decreasing the intensity or removing are identified based on the goal of the treatment plan and the model based on the information tuple. In some cases, upon modification of the treatment plan, the health professional may be notified, via the health professional's computing device 110, that the treatment plan has been modified. In some cases, rather than automatically modifying the treatment plan, the health server 120 proposes modifications to the treatment plan to the health professional and requests approval of the modifications from the heath professional via the health professional's computing device 110.

FIGS. 10A-10B illustrate an example user interface 1000 for identifying data types for a health professional to access, in accordance with some embodiments. The top part of the user interface 1000 is shown in FIG. 10A., and the bottom part of the user interface 1000 is shown in FIG. 10B. The user interface 1000 may be presented at the computing device 140 of the patient and allows the patient to create an authorization (auth) rule for the health professional to access data about the patient from the health data repository 130. As shown, the patient may specify a rule name, a “why string” describing the rule, whether the rule is optional, display flags, permissions, and data types for which the permissions apply. Some examples of data types are illustrated in FIGS. 10A-10B or set forth in Table 1.

TABLE 1
Data types for which permissions may be granted.

| Name | Description |
|---|---|
| Action Plan | A single action plan related object. |
| Advance directive | An advance directive such as a living will. |
| Aerobic profile | A summary of a person's aerobic condition. |
| Allergic episode | A single instance of an allergic reaction. |
| Allergy | A hypersensitivity to an allergen. |
| App-specific | Arbitrary or custom data for use by an |
| information | application. |
| Application data | Information that can be used by an application |
| reference | to render content from another application. |
| Appointment | A medical appointment. |
| Asthma inhaler | An inhaler unit used to treat asthma. |
| Asthma inhaler | A single use of an inhaler. |
| usage | |
| Basic demographic | Defines a set of data about the health |
| information | record that is considered not to be |
| | personally-identifiable. |
| Blood glucose | A single blood glucose reading. |
| Blood oxygen | The percentage of oxygen saturation |
| saturation | in the blood. |
| Blood pressure | A single blood pressure reading. |
| Body composition | A body composition measurement. |
| Body dimension | A body dimension such as waist size |
| | or head circumference. |
| Calorie guideline | A guideline for caloric intake. |
| Cardiac profile | A summary of a person's cardiac condition. |
| Treatment plan | A treatment plan containing tasks and goals. |
| Cholesterol | A single cholesterol reading. |
| Clinical Document | A clinical document architecture. |
| Architecture (CDA) | |
| Comment | A comment associated with another data item. |
| Concern | A concern that a person has about a |
| | condition or life issue. |
| Condition | A medical issue or problem. |
| Contact | A contact such as an emergency |
| | contact, doctor, lawyer, etc. |
| Continuity of Care | A continuity of care document. |
| Document (CCD) | |
| Continuity of Care | A continuity of care record. |
| Record (CCR) | |
| Contraindication | A substance that interacts badly with a |
| | medical condition or drug. |
| Daily dietary intake | The amount of dietary nutrients and |
| | minerals consumed in a day. |
| Daily medication | A record of taking a medication or |
| usage | dietary supplement. |
| Defibrillator | The data from an implantable |
| episode | defibrillator after an episode. |
| Diabetic profile | A summary of a person's diabetic |
| | condition. |
| Discharge | A summary of a discharge from a |
| summary | health provider. |
| Education - MyData | An education file. |
| file (preview) | |
| Education - SIF | An academic record. |
| student academic | |
| record (preview) | |
| Education document | An education document such as an |
| (preview) | assignment or exam. |
| Emotional state | A subjective record of an emotional state. |
| Encounter | A medical encounter such as an annual physical. |
| Exercise | An exercise session such as running or climbing. |
| Exercise samples | A series of data samples from an exercise session. |
| Explanation of | An explanation of benefits received |
| benefits (EOB) | from an insurance plan. |
| Family history | A condition of a relative. |
| Family history | A condition of a relative. |
| condition | |
| Family history | Information about a relative of the |
| person | record owner. |
| File | A file that can be uploaded to a health |
| | record in Microsoft HealthVault. |
| Food & drink | The amount of dietary nutrients and |
| | minerals consumed. |
| Genetic SNP result | A collection of results from a SNP genetic test. |
| Group membership | Memberships of the record owner. |
| Group membership | An activity related to group membership. |
| activity | |
| HbA1C | An HbA1C reading that measures the |
| | amount of glycosylated hemoglobin. |
| Health assessment | The results of a health assessment |
| | such as a diabetes assessment. |
| Health event | A general health event such as the first |
| | time a baby crawls. |
| Health goal | A health goal that defines a target goal |
| | such as steps per day. |
| Health journal entry | An entry of a health journal or diary. |
| Healthcare proxy | A healthcare proxy that appoints an |
| | agent to make medical decisions. |
| Heart rate | A heart rate measurement in beats per minute. |
| Height | A single height measurement. |
| Immunization | An immunization to prevent a disease or |
| | condition. |
| Insight | A single instance of health insight. |
| Insulin injection | An insulin injection used to treat diabetes. |
| Insulin injection | A single use of an insulin injection. |
| usage | |
| Insurance plan | A person or organization that pays for |
| | health and medical bills. |
| Lab results | A series of lab test results. |
| Life goal | A general life goal such as to travel or quit |
| | smoking. |
| Meal definition | A meal that is commonly eaten or a meal |
| | associated with a particular diet plan. |
| Medical | A medical annotation containing |
| annotation | transcribed notes and other information. |
| Medical device | A piece of medical equipment such as |
| | a blood pressure reader or pedometer. |
| Medical image | A study containing medical images. |
| study | |
| Medical problem | A medical problem and diagnosis. |
| Medication | A substance used for the treatment of |
| | a disease or condition. |
| Medication fill | Information related to filling a medication. |
| Menstruation | A single assessment of menstrual flow. |
| Message | A multipart message including |
| | message text and attachments. |
| Microbiology | A microbiology lab test result. |
| lab test result | |
| PAP session | A Positive Airway Pressure (PAP) session. |
| Password-protected | A package that contains a pkcs5v2 encrypted |
| package | blob. |
| Peak flow | A peak flow measurement used to |
| | track lung function. |
| Personal contact | The contact information for the person owning |
| information | a health record in Microsoft HealthVault. |
| Personal demographic | Personal demographic information |
| information | that is considered sensitive in nature. |
| Personal picture | An image that represents the person. |
| Pregnancy | A record of a pregnancy and delivery. |
| Procedure | A medical procedure and results. |
| Question & answer | A question that was asked and the |
| | answers given. |
| Radiology result | The results of a radiology lab test. |
| Respiratory profile | A summary of a person's respiratory condition. |
| Sleep journal entry | A daily journal of activities that impact sleep. |
| Sleep session | A sleep journal entry made in the |
| | morning to reflect on the prior night. |
| Status | The status of an item in the health |
| | record. |
| Vital signs | A set of vital signs such as body temperature. |
| Web link | A link to a web page. |
| Weekly aerobic | A weekly goal for aerobic exercise. |
| exercise goal | |
| Weight | A single weight measurement. |
| Weight goal | A target weight range with an associated |
| | target date. |

### NUMBERED EXAMPLES

Certain embodiments are described herein as numbered examples 1, 2, 3, etc. These numbered examples are provided as examples only and do not limit the subject technology.

Example 1 is a system comprising: one or more processors; and a memory comprising instructions which, when executed by the one or more processors, cause the one or more processors to perform operations comprising: receiving, from a computing device of a health professional, a treatment plan for a patient; receiving, from a plurality of devices associated with the patient, activity data related to the patient; determining whether the activity data is related to compliance with the treatment plan by comparing the activity data with one or more threshold values for the activity data, the one or more threshold values residing in a medical data repository or being provided from the computing device of the health professional; and communicating, in response to determining that the activity data is related to compliance with the treatment plan, the activity data to the computing device of the health professional.

Example 2 is the system of Example 1, the operations further comprising: receiving, from a device from among the plurality of devices associated with the patient, an indicated type of data to provide to the health professional; and determining whether the activity data is associated with the indicated type of data, wherein the activity data is provided to the computing device of the health professional in response to determining that the activity data is associated with the indicated type of data.

Example 3 is the system of any of Examples 1-2, wherein the plurality of devices associated with the patient comprise one or more of: a fitness tracker, a sensor or a computing device configured for manual entry of activity data.

Example 4 is the system of any of Examples 1-3, wherein the treatment plan comprises one or more of: a therapy plan, a disease treatment plan, an exercise plan, a diet plan, or a physiological goal.

Example 5 is the system of any of Examples 1-4, wherein the treatment plan is associated with one or more measurable numerical data points, and wherein the activity data corresponds to the one or more measurable numerical data points.

Example 6 is the system of any of Examples 1-5, wherein determining whether the activity data is related to compliance with the treatment plan comprises determining whether the activity data corresponds to data specified, within the treatment plan, to be reported to the health professional.

Example 7 is the system of any of Examples 1-6, the operations further comprising: receiving, from the computing device of the health professional, an update to the treatment plan generating an updated treatment plan; determining whether the activity data is related to compliance with the updated treatment plan; and communicating, in response to determining that the activity data is related to compliance with the updated treatment plan, the activity data to the computing device of the health professional.

Example 8 is the system of any of Examples 1-7, the operations further comprising: foregoing communicating, in response to determining that the activity data is not related to compliance with the treatment plan, the activity data to the computing device of the health professional,

Example 9 is a non-transitory machine-readable medium comprising instructions which, when executed by one or more processors of a machine, cause the one or more processors to perform operations comprising: receiving, and from a computing device of a health professional, a treatment plan for a patient; receiving, from a plurality of devices associated with the patient, activity data related to the patient; determining whether the activity data is related to compliance with the treatment plan by comparing the activity data with one or more threshold values for the activity data, the one or more threshold values residing in a medical data repository or being provided from the computing device of the health professional; and communicating, in response to determining that the activity data is related to compliance with the treatment plan, the activity data to the computing device of the health professional.

Example 10 is the machine-readable medium of Example 9, the operations further comprising: receiving, from a device from among the plurality of devices associated with the patient, an indicated type of data to provide to the health professional; and determining whether the activity data is associated with the indicated type of data, wherein the activity data is provided to the computing device of the health professional in response to determining that the activity data is associated with the indicated type of data.

Example 11 is the machine-readable medium of any of Examples 9-10, wherein the plurality of devices associated with the patient comprise one or more of: a fitness tracker, a sensor or a computing device configured for manual entry of activity data.

Example 12 is the machine-readable medium of any of Examples 9-11, wherein the treatment plan comprises one or more of: a therapy plan, a disease treatment plan, an exercise plan, a diet plan, or a physiological goal.

Example 13 is the machine-readable medium of any of Examples 9-12, wherein the treatment plan is associated with one or more measurable numerical data points, and wherein the activity data corresponds to the one or more measurable numerical data points.

Example 14 is the machine-readable medium of any of Examples 9-13, wherein determining whether the activity data is related to compliance with the treatment plan comprises determining whether the activity data corresponds to data specified, within the treatment plan, to be reported to the health professional.

Example 15 is the machine-readable medium of any of Examples 9-14, the operations further comprising: receiving, from the computing device of the health professional, an update to the treatment plan generating an updated treatment plan; determining whether the activity data is related to compliance with the updated treatment plan; and communicating, in response to determining that the activity data is related to compliance with the updated treatment plan, the activity data to the computing device of the health professional.

Example 16 is a method comprising: receiving, at a server and from a computing device of a health professional, a treatment plan for a patient; receiving, from a plurality of devices associated with the patient, activity data related to the patient; determining whether the activity data is related to compliance with the treatment plan by comparing the activity data with one or more threshold values for the activity data, the one or more threshold values residing in a medical data repository or being provided from the computing device of the health professional; and communicating, in response to determining that the activity data is related to compliance with the treatment plan, the activity data to the computing device of the health professional.

Example 17 is the method of Example 16, further comprising: receiving, from a device from among the plurality of devices associated with the patient, an indicated type of data to provide to the health professional; and determining whether the activity data is associated with the indicated type of data, wherein the activity data is provided to the computing device of the health professional in response to determining that the activity data is associated with the indicated type of data.

Example 18 is the method of any of Example 16-17, wherein the plurality of devices associated with the patient comprise one or more of: a fitness tracker, a sensor or a computing device configured for manual entry of activity data.

Example 19 is the method of any of Examples 16-18, wherein the treatment plan comprises one or more of: a therapy plan, a disease treatment plan, an exercise plan, a diet plan, or a physiological goal.

Example 20 is the method of any of Example 16-19, wherein the treatment plan is associated with one or more measurable numerical data points, and wherein the activity data corresponds to the one or more measurable numerical data points.

Example 21 is a system comprising: one or more processors; and a memory comprising instructions which, when executed by the one or more processors, cause the one or more processors to perform operations comprising: receiving, from a first computing device of a first health professional, a first treatment plan for a patient; receiving, from a second computing device of a second health professional, a second treatment plan for the patient; receiving, from a plurality of devices associated with the patient, activity data related to the patient; storing, in a data repository, the first treatment plan, the second treatment plan, and the activity data; receiving, from one device from among the plurality of devices associated with the patient, an identification of information to be provided to the first health professional and an identification of information to be provided to the second health professional; identifying first activity data for provision to the first computing device of the first health professional based on the first activity data being related to the first treatment plan and based on the identification of information to be provided to the first health professional, wherein identifying that the first activity data is related to the first treatment plan comprises comparing the first activity data with one or more first threshold values for the first activity data, the one or more first threshold values residing in a medical data repository or being provided from the first computing device of the first health professional; providing the first activity data to the first computing device of the first health professional treatment plan; identifying second activity data for provision to the second computing device of the second health professional based on the second activity data being related to the second treatment plan and based on the identification of information to be provided to the second health professional, wherein identifying that the second activity data is related to the second treatment plan comprises comparing the second activity data with one or more second threshold values for the second activity data, the one or more second threshold values residing in a medical data repository or being provided from the second computing device of the second health professional; and providing the second activity data to the second computing device of the second health professional.

Example 22 is the system of Example 21, the operations further comprising: providing, to the second computing device of the second health professional, the first treatment plan for the patient based on the identification of information to be provided to the second health professional; receiving, from the second computing device of the second health professional, a modification to the first treatment plan; and storing, in the data repository, the modification to the first treatment plan.

Example 23 is the system of Example 22, the operations further comprising: providing, to the first computing device of the first health professional, the modification to the first treatment plan.

Example 24 is the system of any of Examples 21-23, wherein the plurality of devices associated with the patient comprise one or more of: a fitness tracker, a sensor or a computing device configured for manual entry of activity data.

Example 25 is the system of any of Examples 21-24, wherein the first treatment plan or the second treatment plan comprises one or more of: an exercise plan, a diet plan, or a physiological goal.

Example 26 is the system of any of Examples 21-25, wherein the first treatment plan or the second treatment plan is associated with one or more measurable numerical data points, and wherein the activity data corresponds to the one or more measurable numerical data points.

Example 27 is the system of any of Examples 21-26, wherein receiving, from the one device from among the plurality of devices associated with the patient, the identification of information to be provided to the first health professional and the identification of information to be provided to the second health professional comprises: providing for presentation, at the one device, of an interface for identifying the first health professional, the information to be provided to the first health professional, the second health professional, and the information to be provided to the second health professional; and receiving, via the interface, the identification of information to be provided to the first health professional and the identification of information to be provided to the second health professional.

Example 28 is the system of any of Examples 21-27, wherein identifying that the first activity data is related to the first treatment plan comprises applying, to the first activity data, a rule-based analysis provided from the medical data repository or the first computing device of the first health professional.

Example 29 is a non-transitory machine-readable medium comprising instructions which, when executed by one or more processors of a machine, cause the one or more processors to perform operations comprising: receiving, from a first computing device of a first health professional, a first treatment plan for a patient; receiving, from a second computing device of a second health professional, a second treatment plan for the patient; receiving, from a plurality of devices associated with the patient, activity data related to the patient; storing, in a data repository, the first treatment plan, the second treatment plan, and the activity data; receiving, from one device from among the plurality of devices associated with the patient, an identification of information to be provided to the first health professional and an identification of information to be provided to the second health professional; identifying first activity data for provision to the first computing device of the first health professional based on the first activity data being related to the first treatment plan and based on the identification of information to be provided to the first health professional, wherein identifying that the first activity data is related to the first treatment plan comprises comparing the first activity data with one or more first threshold values for the first activity data, the one or more first threshold values residing in a medical data repository or being provided from the first computing device of the first health professional; providing the first activity data to the first computing device of the first health professional; identifying second activity data for provision to the second computing device of the second health professional based on the second activity data being related to the second treatment plan and based on the identification of information to be provided to the second health professional, wherein identifying that the second activity data is related to the second treatment plan comprises comparing the second activity data with one or more second threshold values for the second activity data, the one or more second threshold values residing in a medical data repository or being provided from the second computing device of the second health professional; and providing the second activity data to the second computing device of the second health professional.

Example 30 is the machine-readable medium of Example 29, the operations further comprising: providing, to the second computing device of the second health professional, the first treatment plan for the patient based on the identification of information to be provided to the second health professional; receiving, from the second computing device of the second health professional, a modification to the first treatment plan; and storing, in the data repository, the modification to the first treatment plan.

Example 31 is the machine-readable medium of Example 30, the operations further comprising: providing, to the first computing device of the first health professional, the modification to the first treatment plan.

Example 32 is the machine-readable medium of any of Example 29-31, wherein the plurality of devices associated with the patient comprise one or more of: a fitness tracker, a sensor or a computing device configured for manual entry of activity data.

Example 33 is the machine-readable medium of Example 29-32, wherein the first treatment plan or the second treatment plan comprises one or more of: an exercise plan, a diet plan, or a physiological goal.

Example 34 is the machine-readable medium of any of Examples 29-33, wherein the first treatment plan or the second treatment plan is associated with one or more measurable numerical data points, and wherein the activity data corresponds to the one or more measurable numerical data points.

Example 35 is a method comprising: receiving, from a first computing device of a first health professional, a first treatment plan for a patient; receiving, from a second computing device of a second health professional, a second treatment plan for the patient; receiving, from a plurality of devices associated with the patient, activity data related to the patient; storing, in a data repository, the first treatment plan, the second treatment plan, and the activity data; receiving, from one device from among the plurality of devices associated with the patient, an identification of information to be provided to the first health professional and an identification of information to be provided to the second health professional; identifying first activity data for provision to the first computing device of the first health professional based on the first activity data being related to the first treatment plan and based on the identification of information to be provided to the first health professional, wherein identifying that the first activity data is related to the first treatment plan comprises comparing the first activity data with one or more first threshold values for the first activity data, the one or more first threshold values residing in a medical data repository or being provided from the first computing device of the first health professional; providing the first activity data to the first computing device of the first health professional; identifying second activity data for provision to the second computing device of the second health professional based on the second activity data being related to the second treatment plan and based on the identification of information to be provided to the second health professional, wherein identifying that the second activity data is related to the second treatment plan comprises comparing the second activity data with one or more second threshold values for the second activity data, the one or more second threshold values residing in a medical data repository or being provided from the second computing device of the second health professional; and providing the second activity data to the second computing device of the second health professional.

Example 36 is the method of Example 35, further comprising: providing, to the second computing device of the second health professional, the first treatment plan for the patient based on the identification of information to be provided to the second health professional; receiving, from the second computing device of the second health professional, a modification to the first treatment plan; and storing, in the data repository, the modification to the first treatment plan.

Example 37 is the method of Example 36, further comprising: providing, to the first computing device of the first health professional, the modification to the first treatment plan.

Example 38 is the method of any of Example 35-37, wherein the plurality of devices associated with the patient comprise one or more of: a fitness tracker, a sensor or a computing device configured for manual entry of activity data.

Example 39 is the method of any of Examples 35-38, wherein the first treatment plan or the second treatment plan comprises one or more of: an exercise plan, a diet plan, or a physiological goal.

Example 40 is the method of any of Examples 35-39, wherein the first treatment plan or the second treatment plan is associated with one or more measurable numerical data points, and wherein the activity data corresponds to the one or more measurable numerical data points.

Example 41 is a system comprising: one or more processors; and a memory comprising instructions which, when executed by the one or more processors, cause the one or more processors to perform operations comprising: receiving, at the one or more processors, a medical record of a patient; receiving, from a plurality of devices associated with the patient, activity data and physiological data related to the patient; normalizing the activity data and the physiological data; determining adherence to one or more tasks in a treatment plan based on the medical record, the activity data, and the physiological data by comparing the medical record, the activity data or the physiological data with one or more threshold values, the one or more threshold values residing in a medical data repository or being provided from a computing device of a health professional; selecting, for provision to the health professional, a portion of the medical record, a portion of the activity data, and a portion of the physiological data based on a specification provided by the health professional and permission provided by the patient; and transmitting, to the computing device of the health professional, an indication of adherence to the one or more tasks in the treatment plan, the selected portion of the medical record, the selected portion of the activity data, and the selected portion of the physiological data.

Example 42 is the system of Example 41, the operations further comprising: storing the medical record, the activity data, and the physiological data in a data repository.

Example 43 is the system of any of Examples 41-42, wherein the physiological data comprises one or more of: a weight measurement, a heart rate measurement, or a blood pressure measurement.

Example 44 is the system of any of Examples 41-43, wherein the treatment plan comprises one or more of: an exercise plan, a diet plan, or a physiological goal.

Example 45 is the system of any of Examples 41-44, wherein the treatment plan is associated with one or more measurable numerical data points, and wherein the activity data corresponds to the one or more measurable numerical data points.

Example 46 is the system of any of Example 41-45, wherein the plurality of devices associated with the patient comprise one or more of: a fitness tracker, a sensor or a computing device configured for manual entry of activity data.

Example 47 is the system of Example 41-46, wherein determining adherence to the one or more tasks in the treatment plan based on the medical record, the activity data, and the physiological data comprises: comparing numerical values associated with the one or more tasks with numerical values in the medical record, the activity data, and the physiological data.

Example 48 is a non-transitory machine-readable medium comprising instructions which, when executed by one or more processors of a machine, cause the one or more processors to perform operations comprising: receiving, at the one or more processors, a medical record of a patient; receiving, from a plurality of devices associated with the patient, activity data and physiological data related to the patient; normalizing the activity data and the physiological data; determining adherence to one or more tasks in a treatment plan based on the medical record, the activity data, and the physiological data by comparing the medical record, the activity data or the physiological data with one or more threshold values, the one or more threshold values residing in a medical data repository or being provided from a computing device of a health professional; selecting, for provision to the health professional, a portion of the medical record, a portion of the activity data, and a portion of the physiological data based on a specification provided by the health professional and permission provided by the patient; and transmitting, to the computing device of the health professional, an indication of adherence to the one or more tasks in the treatment plan, the selected portion of the medical record, the selected portion of the activity data, and the selected portion of the physiological data.

Example 49 is the machine-readable medium of Example 48, the operations further comprising: storing the medical record, the activity data, and the physiological data in a data repository.

Example 50 is the machine-readable medium of any of Examples 48-49, wherein the physiological data comprises one or more of: a weight measurement, a heart rate measurement, or a blood pressure measurement.

Example 51 is the machine-readable medium of any of Examples 48-50, wherein the treatment plan comprises one or more of: an exercise plan, a diet plan, or a physiological goal.

Example 52 is the machine-readable medium of any of Examples 48-51, wherein the treatment plan is associated with one or more measurable numerical data points, and wherein the activity data corresponds to the one or more measurable numerical data points.

Example 53 is the machine-readable medium of any of Examples 48-52, wherein the plurality of devices associated with the patient comprise one or more of: a fitness tracker, a sensor or a computing device configured for manual entry of activity data.

Example 54 is the machine-readable medium of any of Examples 48-53, wherein determining adherence to the one or more tasks in the treatment plan based on the medical record, the activity data, and the physiological data comprises: comparing numerical values associated with the one or more tasks with numerical values in the medical record, the activity data, and the physiological data.

Example 55 is a method comprising: receiving, at a server, a medical record of a patient; receiving, from a plurality of devices associated with the patient, activity data and physiological data related to the patient; normalizing the activity data and the physiological data; determining adherence to one or more tasks in a treatment plan based on the medical record, the activity data, and the physiological data by comparing the medical record, the activity data or the physiological data with one or more threshold values, the one or more threshold values residing in a medical data repository or being provided from a computing device of a health professional; selecting, for provision to a health professional, a portion of the medical record, a portion of the activity data, and a portion of the physiological data based on a specification provided by the health professional and permission provided by the patient; and transmitting, to a computing device of the health professional, an indication of adherence to the one or more tasks in the treatment plan, the selected portion of the medical record, the selected portion of the activity data, and the selected portion of the physiological data.

Example 56 is the method of Example 55, further comprising: storing the medical record, the activity data, and the physiological data in a data repository.

Example 57 is the method of any of Examples 55-56, wherein the physiological data comprises one or more of: a weight measurement, a heart rate measurement, or a blood pressure measurement.

Example 58 is the method of any of Examples 55-57, wherein the treatment plan comprises one or more of: an exercise plan, a diet plan, or a physiological goal.

Example 59 is the method of any of Examples 55-58, wherein the treatment plan is associated with one or more measurable numerical data points, and wherein the activity data corresponds to the one or more measurable numerical data points.

Example 60 is the method of any of Examples 55-59, wherein the plurality of devices associated with the patient comprise one or more of: a fitness tracker, a sensor or a computing device configured for manual entry of activity data.

Example 61 is a system comprising: one or more processors; and a memory comprising instructions which, when executed by the one or more processors, cause the one or more processors to perform operations comprising: receiving, at the one or more processors and from a computing device of a health professional, a treatment plan for a patient, the treatment plan comprising a plurality of tasks and a physiological goal; receiving, from a plurality of devices associated with the patient, activity data and physiological data related to the patient; determining, based on the activity data and the physiological data, the patient's compliance with one or more tasks in the treatment plan; and updating the treatment plan based on the patient's compliance with the one or more tasks, the physiological goal, and a model based on an information tuple about the patient.

Example 62 is the system of Example 61, wherein determining the patient's compliance with the one or more tasks comprises determining that the patient is complying with the one or more tasks, and wherein updating the treatment plan comprises increasing an intensity of the one or more tasks.

Example 63 is the system of Example 62, the operations further comprising: identifying the one or more tasks for increasing the intensity based on the physiological goal of the treatment plan and the model based on the information tuple.

Example 64 is the system of any of Examples 61-63, wherein determining the patient's compliance with the one or more tasks comprises determining that the patient is not complying with the one or more tasks, and wherein updating the treatment plan comprises decreasing an intensity of the one or more tasks.

Example 65 is the system of Example 64, the operations further comprising: identifying the one or more tasks for decreasing the intensity based on the physiological goal of the treatment plan and the model based on the information tuple.

Example 66 is the system of any of Examples 61-65, wherein determining the patient's compliance with the one or more tasks comprises determining that the patient is not complying with the one or more tasks, and wherein updating the treatment plan comprises removing at least one of the one or more tasks from the treatment plan.

Example 67 is the system of Example 66, the operations further comprising: identifying the at least one of the one or more tasks from the treatment plan for removal from the treatment plan based on the physiological goal of the treatment plan and the model based on the information tuple.

Example 68 is the system of any of Examples 61-67, wherein the physiological data comprises one or more of: a weight measurement, a heart rate measurement, or a blood pressure measurement.

Example 69 is the system of any of Examples 61-68, wherein the treatment plan comprises one or more of: an exercise plan, a diet plan, or a physiological plan,

Example 70 is the system of any of Examples 61-69, wherein the plurality of devices associated with the patient comprise one or more of: a fitness tracker, a physiological sensor or a computing device configured for manual entry of activity data.

Example 71 is the system of any of Examples 61-70, wherein the information tuple comprises a height, a weight, an age, a gender, and a medical condition.

Example 72 is a non-transitory machine-readable medium comprising instructions which, when executed by one or more processors of a machine, cause the one or more processors to perform operations comprising: receiving, at the one or more processors and from a computing device of a health professional, a treatment plan for a patient, the treatment plan comprising a plurality of tasks and a physiological goal; receiving, from a plurality of devices associated with the patient, activity data and physiological data related to the patient; determining, based on the activity data and the physiological data, the patient's compliance with one or more tasks in the treatment plan; and updating the treatment plan based on the patient's compliance with the one or more tasks, the physiological goal, and a model based on an information tuple about the patient.

Example 73 is the machine-readable medium of Example 72, wherein determining the patient's compliance with the one or more tasks comprises determining that the patient is complying with the one or more tasks, and wherein updating the treatment plan comprises increasing an intensity of the one or more tasks.

Example 74 is the machine-readable medium of Example 73, the operations further comprising: identifying the one or more tasks for increasing the intensity based on the physiological goal of the treatment plan and the model based on the information tuple

Example 75 is the machine-readable medium of any of Examples 72-74, wherein determining the patient's compliance with the one or more tasks comprises determining that the patient is not complying with the one or more tasks, and wherein updating the treatment plan comprises decreasing an intensity of the one or more tasks.

Example 76 is the machine-readable medium of Example 75, the operations further comprising: identifying the one or more tasks for decreasing the intensity based on the physiological goal of the treatment plan and the model based on the information tuple.

Example 77 is the machine-readable medium of any of Examples 72-76, wherein determining the patient's compliance with the one or more tasks comprises determining that the patient is not complying with the one or more tasks, and wherein updating the treatment plan comprises removing at least one of the one or more tasks from the treatment plan.

Example 78 is the machine-readable medium of Example 77, the operations further comprising: identifying the at least one of the one or more tasks from the treatment plan for removal from the treatment plan based on the physiological goal of the treatment plan and the model based on the information tuple.

Example 79 is a method comprising: receiving, at a server and from a computing device of a health professional, a treatment plan for a patient, the treatment plan comprising a plurality of tasks and a physiological goal; receiving, from a plurality of devices associated with the patient, activity data and physiological data related to the patient; determining, based on the activity data and the physiological data, the patient's compliance with one or more tasks in the treatment plan; and updating the treatment plan based on the patient's compliance with the one or more tasks, the physiological goal, and a model based on an information tuple about the patient.

Example 80 is the method of Example 79, wherein the information tuple comprises a height, a weight, an age, aa gender, and a medical condition.

### Components and Logic

Certain embodiments are described herein as including logic or a number of components or mechanisms. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

In some embodiments, a hardware component may be implemented mechanically, electronically, or any suitable combination thereof. For example, a hardware component may include dedicated circuitry or logic that is permanently configured to perform certain operations. For example, a hardware component may be a special-purpose processor, such as a Field-Programmable Gate Array (FPGA) or an Application Specific Integrated Circuit (ASIC). A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations.

Accordingly, the phrase “hardware component” should be understood to encompass a tangible record, be that an record that is physically constructed, permanently configured (e.g., hardwired), or temporarily configured (e.g., programmed) to operate in a certain manner or to perform certain operations described herein. As used herein, “hardware-implemented component” refers to a hardware component. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors.

Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces an API).

The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

### Example Machine and Software Architecture

The components, methods, applications, and so forth described in conjunction with FIGS. 1-10 are implemented in some embodiments in the context of a machine and an associated software architecture. The sections below describe representative software architecture(s and machine (e.g., hardware) architecture(s) that are suitable for use with the disclosed embodiments.

Software architectures are used in conjunction with hardware architectures to create devices and machines tailored to particular purposes. For example, a particular hardware architecture coupled with a particular software architecture will create a mobile device, such as a mobile phone, tablet device, or so forth. A slightly different hardware and software architecture may yield a smart device for use in the “internet of things,” while yet another combination produces a server computer for use within a cloud computing architecture. Not all combinations of such software and hardware architectures are presented here, as those of skill in the art can readily understand how to implement the inventive subject matter in different contexts from the disclosure contained herein.

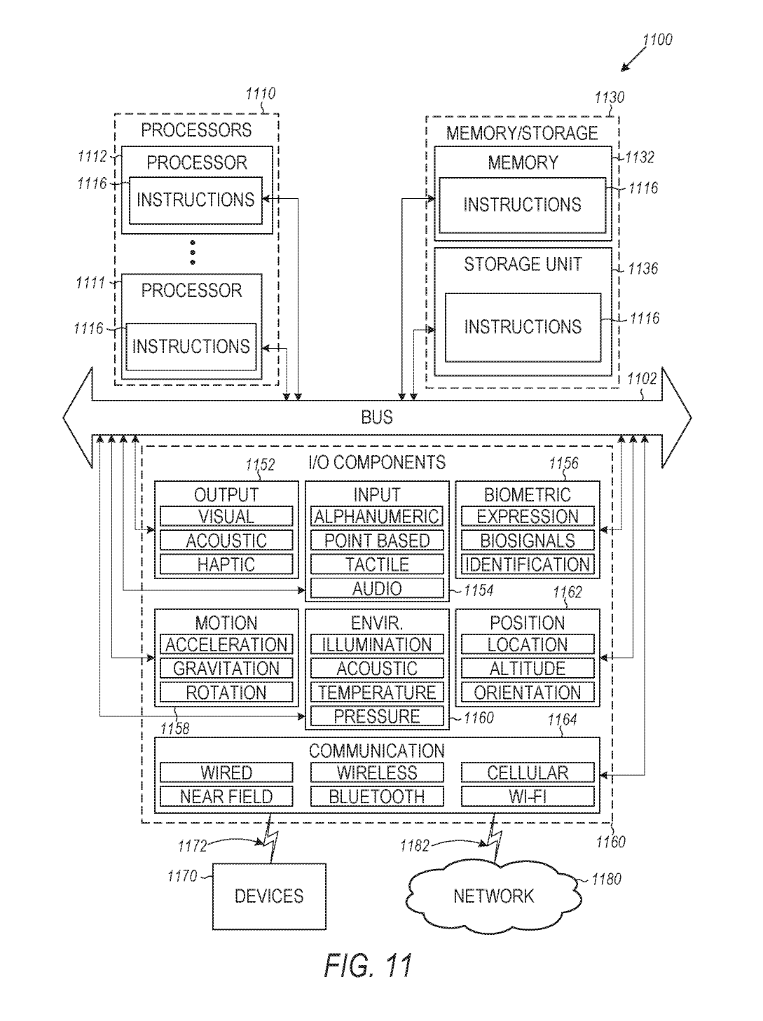

FIG. 11 is a block diagram illustrating components of a machine 1100, according to some example embodiments, able to read instructions from a machine-readable medium (e.g., a machine-readable storage medium) and perform any one or more of the methodologies discussed herein. Specifically, FIG. 11 shows a diagrammatic representation of the machine 1100 in the example form of a computer system, within which instructions 1116 (e.g., software, a program, an application, an applet, an app, or other executable code) for causing the machine 1100 to perform any one or more of the methodologies discussed herein may be executed. The instructions 1116 transform the general, non-programmed machine into a particular machine programmed to carry out the described and illustrated functions in the manner described. In alternative embodiments, the machine 1100 operates as a standalone device or may be coupled (e.g., networked) to other machines. In a networked deployment, the machine 1100 may operate in the capacity of a server machine or a client machine in a server-client network environment, or as a peer machine in a peer-to-peer (or distributed) network environment. The machine 1100 may comprise, but not be limited to, a server computer, a client computer, PC, a tablet computer, a laptop computer, a netbook, a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1116, sequentially or otherwise, that specify actions to be taken by the machine 1100. Further, while only a single machine 1100 is illustrated, the term “machine” shall also be taken to include a collection of machines 1100 that individually or jointly execute the instructions 1116 to perform any one or more of the methodologies discussed herein.

The machine 1100 may include processors 1110, memory/storage 1130, and I/O components 1150, which may be configured to communicate with each other such as via a bus 1102. In an example embodiment, the processors 1110 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1112 and a processor 1114 that may execute the instructions 1116. The term “processor” is intended to include multi-core processors that may comprise two or more independent processors (sometimes referred to as “cores”) that may execute instructions contemporaneously. Although FIG. 11 shows multiple processors 1110, the machine 1100 may include a single processor with a single core, a single processor with multiple cores (e.g., a multi-core processor), multiple processors with a single core, multiple processors with multiples cores, or any combination thereof.

The memory/storage 1130 may include a memory 1132, such as a main memory, or other memory storage, and a storage unit 1136, both accessible to the processors 1110 such as via the bus 1102. The storage unit 1136 and memory 1132 store the instructions 1116 embodying any one or more of the methodologies or functions described herein. The instructions 1116 may also reside, completely or partially, within the memory 1132, within the storage unit 1136, within at least one of the processors 1110 (e.g., within the processor's cache memory), or any suitable combination thereof, during execution thereof by the machine 1100. Accordingly, the memory 1132, the storage unit 1136, and the memory of the processors 1110 are examples of machine-readable media.

As used herein, “machine-readable medium” means a device able to store instructions (e.g., instructions 1116) and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store the instructions 1116. The term “machine-readable medium” shall also be taken to include any medium, or combination of multiple media, that is capable of storing instructions (e.g., instructions 1116) for execution by a machine (e.g., machine 1100), such that the instructions, when executed by one or more processors of the machine (e.g., processors 1110), cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The I/O components 1150 may include a wide variety of components to receive input, provide output, produce output, transmit information, exchange information, capture measurements, and so on. The specific I/O components 1150 that are included in a particular machine will depend on the type of machine. For example, portable machines such as mobile phones will likely include a touch input device or other such input mechanisms, while a headless server machine will likely not include such a touch input device. It will be appreciated that the I/O components 1150 may include many other components that are not shown in FIG. 11. The I/O components 1150 are grouped according to functionality merely for simplifying the following discussion and the grouping is in no way limiting. In various example embodiments, the I/O components 1150 may include output components 1152 and input components 1154. The output components 1152 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1154 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1150 may include biometric components 1156, motion components 1158, environmental components 1160, or position components 1162, among a wide array of other components. For example, the biometric components 1156 may include components to detect expressions (e.g., hand expressions, facial expressions, vocal expressions, body gestures, or eye tracking), measure biosignals (e.g., blood pressure, heart rate, body temperature, perspiration, or brain waves), measure exercise-related metrics (e.g., distance moved, speed of movement, or time spent exercising) identify a person (e.g., voice identification, retinal identification, facial identification, fingerprint identification, or electroencephalogram based identification), and the like. The motion components 1158 may include acceleration sensor components (e.g., accelerometer), gravitation sensor components, rotation sensor components (e.g., gyroscope), and so forth. The environmental components 1160 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components infrared sensors that detect nearby objects), gas sensors (e.g., gas detection sensors to detect concentrations of hazardous gases for safety or to measure pollutants in the atmosphere or other components that may provide indications, measurements, or signals corresponding to a surrounding physical environment. The position components 1162 may include location sensor components (e.g., a Global Position System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1150 may include communication components 1164 operable to couple the machine 1100 to a network 1180 or devices 1170 via a coupling 1182 and a coupling 1172, respectively. For example, the communication components 1164 may include a network interface component or other suitable device to interface with the network 1180 In further examples, the communication components 1164 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1170 may be another machine or any of a wide variety of peripheral devices (e.g., a peripheral device coupled via a USB).

Moreover, the communication components 1164 may detect identifiers or include components operable to detect identifiers. For example, the communication components 1164 may include Radio Frequency Identification (RFID) tag reader components, NFC smart tag detection components, optical reader components, or acoustic detection components (e.g., microphones to identify tagged audio signals). In addition, a variety of information may be derived via the communication components 1164, such as location via Internet Protocol (IP) geolocation, location via Wi-Fi® signal triangulation, location via detecting an NFC beacon signal that may indicate a particular location, and so forth.

In various example embodiments, one or more portions of the network 1180 may be an ad hoc network, an intranet, an extranet, a virtual private network (VPN), a local area network (LAN), a wireless LAN (WLAN), a WAN, a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, the network 1180 or a portion of the network 1180 may include a wireless or cellular network and the coupling 1182 may be a Code Division Multiple Access (CDMA) connection, a Global System for Mobile communications (GSM) connection, or another type of cellular or wireless coupling. In this example, the coupling 1182 may implement any of a variety of types of data transfer technology, such as Single Carrier Radio Transmission Technology (1xRTT), Evolution-Data Optimized (EVDO) technology, General Packet Radio Service (CPRS) technology, Enhanced Data rates for GSM Evolution (EDGE) technology, third Generation Partnership Project (3GPP) including 3G. fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1116 may he transmitted or received over the network 1180 using a transmission medium via a network interface device (e.g., a network interface component included in the communication components 1164) and utilizing any one of a number of well-known transfer protocols (e.g., HTTP). Similarly, the instructions 1116 may be transmitted or received using a transmission medium via the coupling 1172 (e.g., a peer-to-peer coupling) to the devices 1170. The term “transmission medium” shall be taken to include any intangible medium that is capable of storing, encoding, or carrying the instructions 1116 for execution by the machine 1100, and includes digital or analog communications signals or other intangible media to facilitate communication of such software.

What is claimed is:

1. A system comprising:
one or more processors; and
a memory comprising instructions which, when executed by the one or more processors, cause the one or more processors to perform operations comprising:
receiving, at the one or more processors, a medical record of a patient;
receiving, from a plurality of devices associated with the patient, activity data and physiological data related to the patient;
normalizing the activity data and the physiological data;
determining adherence to one or more tasks in a treatment plan based on the medical record, the activity data, and the physiological data by comparing the medical record, the activity data or the physiological data with one or more threshold values, the one or more threshold values residing in a medical data repository or being provided from a computing device of a health professional;
selecting, for provision to the health professional, a portion of the medical record, a portion of the activity data, and a portion of the physiological data based on a specification provided by the health professional and permission provided by the patient; and
transmitting, to the computing device of the health professional, an indication of adherence to the one or more tasks in the treatment plan, the selected portion of the medical record, the selected portion of the activity data, and the selected portion of the physiological data.

2. The system of claim 1, the operations further comprising:
storing the medical record, the activity data, and the physiological data in a data repository.

3. The system of claim 1, wherein the physiological data comprises one or more of: a weight measurement, a heart rate measurement, or a blood pressure measurement.

4. The system of claim I, wherein the treatment plan comprises one or more of: an exercise plan, a diet plan, or a physiological goal.

5. The system of claim I, wherein the treatment plan is associated with one or more measurable numerical data points, and wherein the activity data corresponds to the one or more measurable numerical data points.

6. The system of claim 1, wherein the plurality of devices associated with the patient comprise one or more of: a fitness tracker, a sensor or a computing device configured for manual entry of activity data.

7. The system of claim I., wherein determining adherence to the one or more tasks in the treatment plan based on the medical record, the activity data, and the physiological data comprises:
comparing numerical values associated with the one or more tasks with numerical values in the medical record, the activity data, and the physiological data.

8. A non-transitory machine-readable medium comprising instructions which, when executed by one or more processors of a machine, cause the one or more processors to perform operations comprising:
receiving, at the one or more processors, a medical record of a patient;
receiving, from a plurality of devices associated with the patient, activity data and physiological data related to the patient;
normalizing the activity data and the physiological data;
determining adherence to one or more tasks in a treatment plan based on the medical record, the activity data, and the physiological data by comparing the medical record, the activity data or the physiological data with one or more threshold values, the one or more threshold values residing in a medical data repository or being provided from a computing device of a health professional;
selecting, for provision to the health professional, a portion of the medical record, a portion of the activity data, and a portion of the physiological data based on a specification provided by the health professional and permission provided by the patient, and
transmitting, to the computing device of the health professional, an indication of adherence to the one or more tasks in the treatment plan, the selected portion of the medical record, the selected portion of the activity data, and the selected portion of the physiological data.

9. The machine-readable medium of claim 8, the operations further comprising:
storing the medical record, the activity data, and the physiological data in a data repository.

10. The machine-readable medium of claim 8, wherein the physiological data comprises one or more of: a weight measurement, a heart rate measurement, or a blood pressure measurement.

11. The machine-readable medium of claim 8, wherein the treatment plan comprises one or more of: an exercise plan, a diet plan, or a physiological goal.

12. The machine-readable medium of claim 8, wherein the treatment plan is associated with one or more measurable numerical data points, and wherein the activity data corresponds to the one or more measurable numerical data points.

13. The machine-readable medium of claim 8, wherein the plurality of devices associated with the patient comprise one or more of: a fitness tracker, a sensor or a computing device configured for manual entry of activity data.

14. The machine-readable medium of claim 8, wherein determining adherence to the one or more tasks in the treatment plan based on the medical record, the activity data, and the physiological data comprises:
comparing numerical values associated with the one or more tasks with numerical values in the medical record, the activity data, and the physiological data.

15. A method comprising:
receiving, at a server, a medical record of a patient,
receiving, from a plurality of devices associated with the patient, activity data and physiological data related to the patient;
normalizing the activity data and the physiological data;
determining adherence to one or more tasks in a treatment plan based on the medical record, the activity data, and the physiological data by comparing the medical record, the activity data or the physiological data with one or more threshold values, the one or more threshold values residing in a medical data repository or being provided from a computing device of a health professional;
selecting, for provision to a health professional, a portion of the medical record, a portion of the activity data, and a portion of the physiological data based on a specification provided by the health professional and permission provided by the patient; and
transmitting, to a computing device of the health professional, an indication of adherence to the one or more tasks in the treatment plan, the selected portion of the medical record, the selected portion of the activity data, and the selected portion of the physiological data.

16. The method of claim 15, further comprising:
storing the medical record, the activity data, and the physiological data in a data repository.

17. The method of claim 15, wherein the physiological data comprises one or more of: a weight measurement, a heart rate measurement, or a blood pressure measurement.

18. The method of claim 15, wherein the treatment plan comprises one or more of: an exercise plan, a diet plan, or a physiological goal.

19. The method of claim 15, wherein the treatment plan is associated with one or more measurable numerical data points, and wherein the activity data corresponds to the one or more measurable numerical data points.

20. The method of claim 15, wherein the plurality of devices associated with the patient comprise one or more of: a fitness tracker, a sensor or a computing device configured for manual entry of activity data.
